(19) United States
(12) Patent Application Publication
(10) Pub. No.: US 2024/0181080 A1
(43) Pub. Date: Jun. 6, 2024

(54) METHOD FOR CONTROLLING HETEROGENEITY OF VASCULAR ENDOTHELIAL CELLS BY UTILIZING SYMPATHETIC NERVES

(71) Applicant: SHENZHEN INSTITUTES OF ADVANCED TECHNOLOGY CHINESE ACADEMY OF SCIENCES, Shenzhen (CN)

(72) Inventors: Yingzi ZHAO, Shenzhen (CN); Botai LI, Shenzhen (CN); Dashuang GAO, Shenzhen (CN); Fan YANG, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 18/434,358

(22) Filed: Feb. 6, 2024

Related U.S. Application Data
(63) Continuation of application No. PCT/CN2021/113818, filed on Aug. 20, 2021.

(51) Int. Cl. A61K 48/00; A61K 31/5513; A61K 38/17; A61P 19/08; C12N 15/86

(52) U.S. Cl. CPC A61K 48/005; A61K 31/5513; A61K 38/177; A61P 19/08; C12N 15/86; C12N 2740/15043; C12N 2750/14143

(57) ABSTRACT

Disclosed in the present invention is A method for controlling heterogeneity of vascular endothelial cells by utilizing sympathetic nerves is provided, comprising expressing an artificial designer protein receptor exclusively activated by a designer drug in neuron cells, and then activating the artificial designer protein receptor by the designer drug, thereby activating or inhibiting neuronal activity to achieve control of the heterogeneity of the vascular endothelial cells. The method enables local modulation of the sympathetic nervous activity, thereby controlling the heterogeneity of the vascular endothelial cells, and can achieve bidirectional modulation. According to the present invention, the heterogeneity of bone vascular endothelial cells is controlled by manipulating the sympathetic nerves, such that the subtype of the bone vascular endothelial cells is controlled, thereby affecting the bone mineral density coupled therewith. The present invention has important clinical significance in the field of treatment or prevention of osteoporosis.

### TECHNICAL FIELD

The present invention relates to bone and blood vessels, and particularly to a method for controlling heterogeneity of vascular endothelial cells by utilizing sympathetic nerves.

### BACKGROUND

Vascular endothelial cells are a monolayer of cells distributed on the inner wall of blood vessels with significant heterogeneity, and through dense capillary branching, the vascular endothelium establishes communication with almost all cells within each organ. The heterogeneity of the vascular endothelial cells is manifested as phenotypic heterogeneity and functional heterogeneity. Specifically, the endothelial cells are located in different organs and different positions of the same organ, and the vascular endothelium has significant morphological specificity; meanwhile, the vascular endothelial cells in different organs have different transcription factor clusters expressed corresponding to different organs and needs, and secrete different vascular secretion factors to support the physiological functions of organs. The heterogeneity of the endothelial cells is associated with intrinsic factors modified by cellular genes and extrinsic factors induced by the extracellular microenvironment.

The intraosseous vascular endothelium has abundant heterogeneity. H-subtype bone vascular endothelium, one of the subtypes of the vascular endothelium, primarily presents in an arch column shape, is distributed near the bone growth plate and endosteum, and is a subtype of endothelial cells with highly active growth and metabolism. It is coupled with the bone metabolism through Notch signaling pathways to promote osteosis. As age increases, the expression of two marker antibodies, CD31 and EMCN, in the H-subtype endothelial cells decreases, and H-subtype endothelium transforms into L-subtype endothelium that no longer retains the properties of highly active growth and metabolism and promotion of osteosis, such that the bone mass begins to gradually lose, resulting in osteoporosis. Studies have shown that hypoxia-inducible factors, Notch signaling pathways, blood flow, platelet-derived growth factors secreted by osteoclast precursors, and the adaptor protein Schnurri311 secreted by osteoblasts, and the like are associated with the in-vivo levels of the H-subtype endothelial cells. Currently, in the prior art, the subtypes of the bone vascular endothelial cells are controlled mainly by genetic modification or taking drugs, thereby improving the bone mineral density. For example, the drug deferoxamine mesylate promotes the reverse transformation of L-subtype vascular endothelium into H-subtype vascular endothelium, thereby increasing the bone mineral density in aged C57BL/6J male mice.

There is currently no method for controlling the heterogeneity of the vascular endothelial cells by utilizing sympathetic nerves. Therefore, it is of great clinical significance to study a method for controlling the heterogeneity of the vascular endothelial cells by utilizing the sympathetic nerves.

### SUMMARY

In view of the deficiencies in the prior art, the present invention aims to provide a method for controlling heterogeneity of vascular endothelial cells by utilizing sympathetic nerves.

In pharmacogenetics, genetically modified G protein-coupled receptors, also known as “designer receptors exclusively activated by designer drugs” (DREADDs), are utilized, and the designer drugs that do not participate in normal physiological activities of the body are injected to activate the artificial designer protein receptors, thereby selectively activating or inhibiting neuronal activity.

According to the present invention, by utilizing the principles of pharmacogenetics, the heterogeneity of the vascular endothelial cells, particularly the heterogeneity of bone vascular endothelial cells, is controlled by manipulating the sympathetic nerves.

According to a first aspect of the present invention, provided is a method for controlling heterogeneity of vascular endothelial cells by utilizing sympathetic nerves, comprising expressing an artificial designer protein receptor exclusively activated by a designer drug in neuron cells, and then activating the artificial designer protein receptor by the designer drug, thereby activating or inhibiting neuronal activity to achieve control of the heterogeneity of the vascular endothelial cells,
wherein the artificial designer protein receptor is a protein receptor that can activate or inhibit the neuronal activity under the activation effect of the designer drug.

In the method described above, the artificial designer protein receptor is hM4Di or hM3Dq;
the designer drug is clozapine N-oxide or deschloroclozapine, and preferably, the designer drug is clozapine N-oxide;
the neuron cells are neuron cells in a mammal, wherein preferably, the mammal is a young mammal, and more preferably, the mammal is a mouse aged 3-14 weeks.

In the method described above, preferably, the vascular endothelial cells are bone vascular endothelial cells.

In the method described above, when the artificial designer protein receptor is hM4Di, the designer drug activates hM4Di to inhibit the neuronal activity and reduce the number of H-subtype endothelial cells.

In the method described above, when the artificial designer protein receptor is hM3Dq, the designer drug activates hM3Dq to activate the neuronal activity and increase the number of H-subtype endothelial cells.

According to a second aspect of the present invention, provided is a method for changing bone mineral density or bone mass, comprising expressing an artificial designer protein receptor exclusively activated by a designer drug in intraosseous neuron cells, and then activating the artificial designer protein receptor by the designer drug to activate or inhibit neuronal activity and modulate the number of H-subtype endothelial cells, thereby changing the bone mineral density or bone mass,
wherein the artificial designer protein receptor is a protein receptor that can activate or inhibit the neuronal activity under the activation effect of the designer drug. The designer drug activates the artificial designer protein receptor to activate or inhibit the neuronal activity and modulate the number of the H-subtype endothelial cells, thereby changing the bone mineral density or bone mass.

In the method described above, the artificial designer protein receptor is hM4Di or hM3Dq;
the designer drug is clozapine N-oxide or deschloroclozapine, and preferably, the designer drug is clozapine N-oxide;
the intraosseous neuron cells are intraosseous neuron cells in a mammal, wherein preferably, the mammal is a young mammal, and more preferably, the mammal is a mouse aged 3-14 weeks.

In the method described above, when the artificial designer protein receptor is hM4Di, the designer drug activates hM4Di to inhibit the neuronal activity and reduce the number of the H-subtype endothelial cells, thereby reducing the bone mineral density or bone mass.

In the method described above, when the artificial designer protein receptor is hM3Dq, the designer drug activates hM3Dq to activate the neuronal activity and increase the number of the H-subtype endothelial cells, thereby increasing the bone mineral density or bone mass.

According to a third aspect of the present invention, provided is a method for treating or preventing osteoporosis, comprising expressing an artificial designer protein receptor exclusively activated by a designer drug in intraosseous neuron cells, and then activating the artificial designer protein receptor by the designer drug to activate neuronal activity and increase the number of H-subtype endothelial cells, thereby achieving the treatment or prevention of osteoporosis,
wherein the artificial designer protein receptor is a protein receptor that can activate the neuronal activity under the activation effect of the designer drug. The designer drug activates the artificial designer protein receptor to activate the neuronal activity and increase the number of the H-subtype endothelial cells, thereby achieving the treatment or prevention of osteoporosis.

In the method described above, the artificial designer protein receptor is hM3Dq;
the designer drug is clozapine N-oxide or deschloroclozapine, and preferably, the designer drug is clozapine N-oxide;
the intraosseous neuron cells are intraosseous neuron cells in a mammal, wherein preferably, the mammal is a young mammal, and more preferably, the mammal is a mouse aged 3-14 weeks.

According to a fourth aspect of the present invention, provided is a pharmaceutical composition for changing bone mass or bone mineral density, comprising an artificial designer protein receptor exclusively activated by a designer drug and/or a designer drug, or comprising a virus carrying a chemical genetic gene and/or a designer drug activating a chemical genetic gene, wherein the chemical genetic gene is a gene encoding an artificial designer protein receptor exclusively activated by a designer drug;
the artificial designer protein receptor is a protein receptor that can activate or inhibit the neuronal activity under the activation effect of the designer drug.

In the pharmaceutical composition described above, the virus is an adeno-associated virus or a lentivirus, and preferably, the virus is an adeno-associated virus;
the artificial designer protein receptor is hM4Di or hM3Dq;
the designer drug is clozapine N-oxide or deschloroclozapine, and preferably, the designer drug is clozapine N-oxide;
preferably, a target subject for the pharmaceutical composition is a mammal, wherein preferably, the mammal is a young mammal, and more preferably, the mammal is a mouse aged 3-14 weeks.

In the pharmaceutical composition described above, when the artificial designer protein receptor is hM4Di, the designer drug activates hM4Di to inhibit the neuronal activity and reduce the number of H-subtype endothelial cells, thereby reducing the bone mineral density or bone mass.

In the pharmaceutical composition described above, when the artificial designer protein receptor is hM3Dq, the designer drug activates hM3Dq to activate the neuronal activity and increase the number of H-subtype endothelial cells, thereby increasing the bone mineral density or bone mass.

According to a fifth aspect of the present invention, provided is a pharmaceutical composition for treating or preventing osteoporosis, comprising an artificial designer protein receptor exclusively activated by a designer drug and/or a designer drug, or comprising a virus carrying a chemical genetic gene and/or a designer drug activating a chemical genetic gene, wherein the chemical genetic gene is a gene encoding an artificial designer protein receptor exclusively activated by a designer drug;
the artificial designer protein receptor is a protein receptor that can activate the neuronal activity under the activation effect of the designer drug.

In the pharmaceutical composition described above, the virus is an adeno-associated virus or a lentivirus, and preferably, the virus is an adeno-associated virus;
the artificial designer protein receptor is hM3Dq;
the designer drug is clozapine N-oxide or deschloroclozapine, and preferably, the designer drug is clozapine N-oxide;
preferably, a target subject for the pharmaceutical composition is a mammal, wherein preferably, the mammal is a young mammal, and more preferably, the mammal is a mouse aged 3-14 weeks.

The pharmaceutical composition described above of the present invention further comprises a pharmaceutically acceptable carrier.

According to a sixth aspect of the present invention, provided is a kit, comprising the pharmaceutical composition for changing the bone mass or bone mineral density described above, or the pharmaceutical composition for treating or preventing osteoporosis described above.

According to a seventh aspect of the present invention, provided is an animal model obtained by the method for controlling the heterogeneity of the vascular endothelial cells by utilizing the sympathetic nerves, the method for changing the bone mineral density or bone mass, or the method for treating or preventing osteoporosis, wherein preferably, the animal model is a mouse model of osteoporosis.

In the technical solutions of the present invention, the designer drug can be taken by a simple route, such as intramuscular injection, intravenous injection, intraperitoneal injection, subcutaneous injection, or oral administration.

The present invention has the following beneficial effects.
1. According to the method for controlling the heterogeneity of the vascular endothelial cells by utilizing the sympathetic nerves provided in the present invention, the artificial designer protein receptor exclusively activated by the designer drug is expressed in the neuron cells, and then the artificial designer protein receptor is activated by the designer drug, thereby activating or inhibiting the neuronal activity to achieve the control of the heterogeneity of the vascular endothelial cells. The method enables local modulation of the sympathetic nervous activity, thereby controlling the heterogeneity of the vascular endothelial cells, and can achieve bidirectional modulation.
2. Bone marrow mesenchymal stem cells are continuously differentiated into osteoprogenitor cells to further form osteoblasts so as to participate in bone formation. The whole bone formation requires the participation of blood vessels, and the formation of the blood vessels requires the participation of endothelial cells. Intraosseous H-subtype blood vessels are accompanied by the osteoprogenitor cells, and the H-subtype blood vessels are mainly shown to be positive for two marker antibodies, CD31 and RMCN, in the endothelial cells. As the expression of the two marker antibodies, CD31 and EMCN, in the H-subtype endothelial cells decreases, H-subtype endothelium transforms into L-subtype endothelium that no longer retains the properties of highly active growth and metabolism and promotion of osteosis, such that the bone mass begins to gradually lose. According to the method for changing the bone mineral density or bone mass provided in the present invention, by utilizing the principles of pharmacogenetics, the heterogeneity of the bone vascular endothelial cells is controlled by manipulating the sympathetic nerves, such that the subtype of the bone vascular endothelial cells is controlled, thereby affecting the bone mineral density coupled therewith. The method has important clinical significance in the field of treatment or prevention of osteoporosis.
3. There are H-subtype bone vascular endothelial cells in the bones of young mammals, but as age increases, the H subtype gradually transforms into the L subtype in adult organisms, resulting in the loss of the function of promoting bone formation. During the growth and development of young mammals, various cells in various organs still have the potential to grow and differentiate. By activating or inhibiting the neuronal activity, the heterogeneity of the vascular endothelial cells is modulated, such that the endothelial cells release endothelial secretion factors that act on surrounding bone cells to modulate the growth and development of the cells. According to the method for treating or preventing osteoporosis provided in the present invention, the heterogeneity of the bone vascular endothelial cells is controlled by manipulating the sympathetic nerves, such that the number of the H-subtype bone vascular endothelial cells is increased, thereby affecting the bone mineral density coupled therewith, promoting the bone formation, and thus achieving the treatment or prevention of osteoporosis.

### BRIEF DESCRIPTION OF DRAWINGS

FIG. 1 shows fluorescence microscope images of bone vascular endothelial cells in the epiphyseal end of the femur of a mouse in Example 1, wherein A of FIG. 1 is for a control group and B of FIG. 1 is for an experimental group.

FIG. 2 shows microCT images of the trabecular bone at the epiphyseal end of the femur of the mouse in Example 1, wherein A of FIG. 2 is for the control group and B of FIG. 2 is for the experimental group.

FIG. 3 shows statistical charts for the changes in the bone mass and morphology of the trabecular bone in Example 1, wherein A of FIG. 3 is for the trabecular bone mineral density (Tb.BMD), B of FIG. 3 is for the trabecular bone thickness (Tb.Th), C of FIG. 3 is for the trabecular bone volume fraction (BV/TV), and D of FIG. 3 is for the trabecular bone number (Tb.N); *: p<0.05, **: p<0.01.

FIG. 4 shows fluorescence microscope images of bone vascular endothelial cells in the epiphyseal end of the femur of a mouse in Example 2, wherein A of FIG. 4 is for a control group and B of FIG. 4 is for an experimental group.

FIG. 5 shows microCT images of the trabecular bone at the epiphyseal end of the femur of the mouse in Example 2, wherein A of FIG. 5 is for the control group and B of FIG. 5 is for the experimental group.

FIG. 6 shows statistical charts for the changes in the bone mass and morphology of the trabecular bone in Example 2, wherein A of FIG. 6 is for the trabecular bone mineral density (Tb.BMD), B of FIG. 6 is for the trabecular bone thickness (Tb.Th), C of FIG. 6 is for the trabecular bone volume fraction (BV/TV), and D of FIG. 6 is for the trabecular bone number (Tb.N); *: p<0.05, **: p<0.01.

### DETAILED DESCRIPTION OF THE EMBODIMENTS

In order to understand the present invention more clearly, the present invention will be further described with reference to the following examples and drawings. The examples are given for the purpose of illustration only and are not intended to limit the present invention in any way. In the examples, all of the reagents and starting materials are commercially available, and the experimental methods without specifying the specific conditions are conventional methods with conventional conditions well known in the art, or conditions suggested by the instrument manufacturer.

### Terminology

hM4Di and hM3Dq are artificial designer protein receptors, which are mutated human muscarinic receptors. The mutated human M4 muscarinic receptor is called hM4Di, which binds to CNO to inhibit neurons; the mutated M3 muscarinic receptor is called hM3Dq, which binds to CNO to activate neurons.

Clozapine N-oxide (CNO) is an artificial designer drug in the DREADD system.

Deschloroclozapine (DCZ), which is extremely similar in structure to clozapine, has a higher affinity for hM4Di and hM3Dq.

AAV means adenovirus.

TH-Cre transgenic mice, in combination with a virus carrying a plasmid with a DIO element, are capable of specifically expressing hM4Di or hM3Dq in sympathetic nerves.

CD31 and EMCN are markers for H-subtype bone vascular endothelium; CD31 is marked with green fluorescence, and EMCN is marked with red fluorescence.

### Example 1

### 1. Construction of Adenovirus

An hM3Dq gene, a mCherry gene and a neuron-specific promoter were constructed on a pAAV plasmid to give pAAV-hSyn-DIO-hM3D(Gq)-mCherry.

AmCherry gene and a neuron-specific promoter were constructed on a pAAV plasmid to give pAAV-hSyn-DIO-mCherry.

The constructed plasmids were each transfected into 293T cells. After the transfection was completed, the viral particles were enriched to give an adenovirus carrying pAAV-hSyn-DIO-hM3D(Gq)-mCherry or pAAV-hSyn-DIO-mCherry with a viral titer of 1013 vg/ML.

In this example, the construction of the adenoviral vectors was carried out using conventional methods and conditions in the art.

2. Expression of hM3Dq Protein

A TH-Cre transgenic mouse aged 4 weeks was selected and anesthetized, and then the skin and muscle on the femur of the mouse were carefully parted. A hole was made in the femur using a small needle to reach the marrow cavity. Using a microsyringe, 1 μL of pAAV-hSyn-DIO-hM3D(Gq)-mCherry with a viral titer of about 1013 vg/ML was slowly injected into the long bone marrow cavity of the TH-Cre mouse on one side of the femur in the lower limb through the small hole, serving as an experimental group (recorded as hM3Dq); on the other side, pAAV-hSyn-DIO-mCherry was injected, serving as a control group (recorded as mCherry). After the injection was completed, the needle was left in place for a period of time to allow the tissue to fully absorb the virus-containing injection. The syringe was then carefully withdrawn, and the small hole was sealed with bone wax. The muscle and skin were sutured, and anti-inflammatory and analgesic medication was applied to the wound.

### 3. Injection of Designer Drug

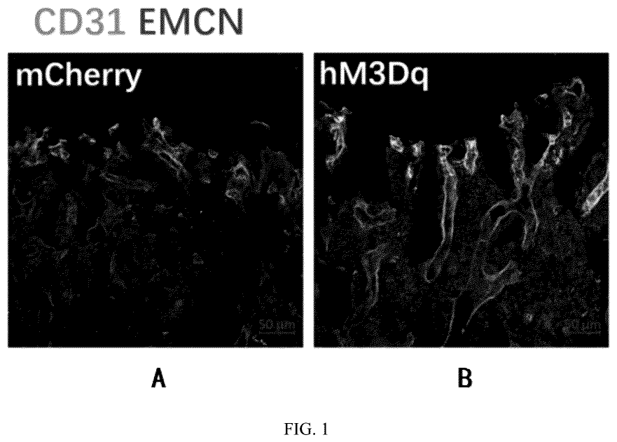

After recovery of the mouse and 6 weeks of expression of the gene carried by the virus, CNO was injected (at a dose to achieve a concentration of 1 mg/kg in the mouse) every 48 h for 4 consecutive weeks. After the 4-week injection, the femur was harvested, and data were obtained. The results are shown in FIGS. 1-3. Data were expressed as (Means±S.E.M.); P<0.05 was considered a significant difference.

FIG. 1 shows fluorescence microscopic images of bone vascular endothelial cells in the epiphyseal end of the femur of the mouse. CD31 is marked with green fluorescence, and EMCN is marked with red fluorescence; both are markers for H-subtype bone vascular endothelium. The stronger the fluorescence intensity, the more complete the vascular morphology, indicating greater expression of CD31 and EMCN, i.e., the higher number of the H-subtype bone vascular endothelial cells. A of FIG. 1 is for the control group and B of FIG. 1 is for the experimental group. Compared with the control group injected with the adenovirus not carrying hM3Dq gene, the sympathetic nerve activity on the side injected with the adenovirus carrying hM3Dq gene was activated, resulting in an increase in the number of the H-subtype bone vascular endothelial cells near the long bone growth plate.

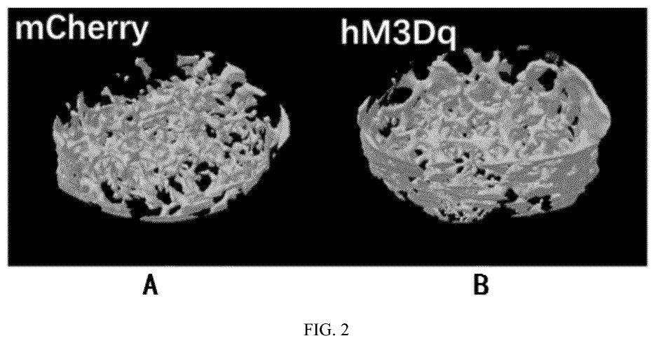

FIG. 2 shows microCT images of the trabecular bone at the epiphyseal end of the femur of the mouse, wherein A of FIG. 2 is for the control group and B of FIG. 2 is for the experimental group. Compared with the control group injected with the adenovirus not carrying hM3Dq gene, the experimental group injected with the adenovirus carrying hM3Dq gene showed an increase in the bone mass of the trabecular bone after activation of the sympathetic nerves.

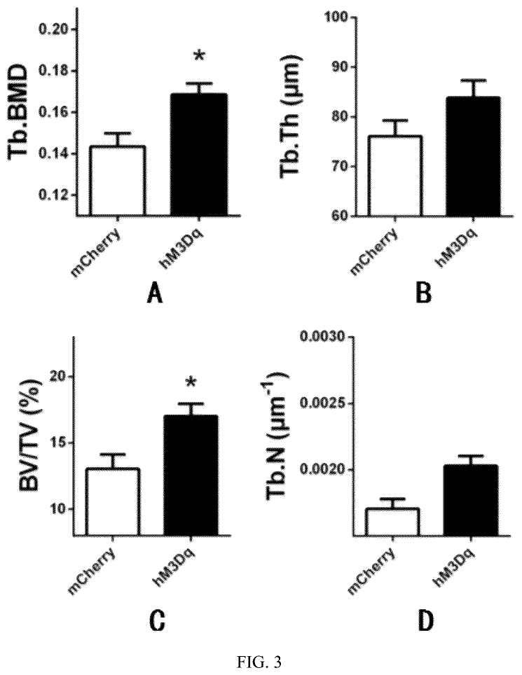

FIG. 3 shows statistical charts for the changes in the bone mass and morphology of the trabecular bone, wherein A of FIG. 3 is for the trabecular bone mineral density, B of FIG. 3 is for the trabecular bone thickness, C of FIG. 3 is for the trabecular bone volume fraction, and D of FIG. 3 is for the trabecular bone number. Compared with the control group, the experimental group showed a significant increase in both trabecular bone mineral density and trabecular bone volume fraction, with a trend of increase in the trabecular bone thickness and trabecular bone number.

As can be seen from the experimental results shown in FIGS. 1-3, by expressing hM3Dq in the intraosseous neuron cells and injecting the designer drug CNO, the sympathetic nerve activity could be activated, such that the H-subtype bone vascular endothelial cells near the long bone growth plate were increased, resulting in a rise in the bone mass of the trabecular bone.

### Example 2

### 1. Construction of Adenovirus

An hM4Di gene, a mCherry gene and a neuron-specific promoter were constructed on a pAAV plasmid to give pAAV-hSyn-DIO-hM4Di-mCherry.

AmCherry gene and a neuron-specific promoter were constructed on a pAAV plasmid to give pAAV-hSyn-DIO-mCherry.

The constructed plasmids were each transfected into 293T cells. After the transfection was completed, the viral particles were enriched to give an adenovirus carrying pAAV-hSyn-DIO-hM4Di-mCherry or pAAV-hSyn-DIO-mCherry with a viral titer of 1013 vg/ML.

In this example, the construction of the adenoviral vectors was carried out using conventional methods and conditions in the art.

2. Expression of hM4Di Protein

A TH-Cre transgenic mouse aged 4 weeks was selected and anesthetized, and then the skin and muscle on the femur of the mouse were carefully parted. A hole was made in the femur using a small needle to reach the marrow cavity. Using a microsyringe, 1 μL of pAAV-hSyn-DIO-hM4Di-mCherry with a viral titer of about 1013 vg/ML was slowly injected into the long bone marrow cavity of the TH-Cre mouse on one side of the femur in the lower limb through the small hole, serving as an experimental group (recorded as hM4Di); on the other side, pAAV-hSyn-DIO-mCherry was injected, serving as a control group (recorded as mCherry). After the injection was completed, the needle was left in place for a period of time to allow the tissue to fully absorb the virus-containing injection. The syringe was then carefully withdrawn, and the small hole was sealed with bone wax. The muscle and skin were sutured, and anti-inflammatory and analgesic medication was applied to the wound.

### 3. Injection of Designer Drug

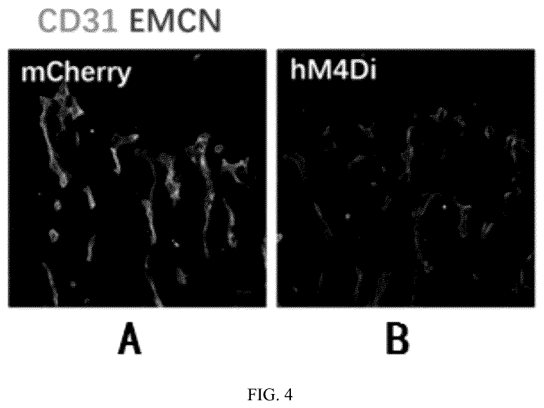

After recovery of the mouse and 6 weeks of expression of the gene carried by the virus, CNO was injected (at a dose to achieve a concentration of 1 mg/kg in the mouse) every 48 h for 4 consecutive weeks. After the 4-week injection, the femur was harvested, and data were obtained. The results are shown in FIGS. 4-6. Data were expressed as (Means±S.E.M.); P<0.05 was considered a significant difference.

FIG. 4 shows fluorescence microscopic images of bone vascular endothelial cells in the epiphyseal end of the femur of the mouse. CD31 is marked with green fluorescence, and EMCN is marked with red fluorescence; both are markers for H-subtype bone vascular endothelium. A of FIG. 4 is for the control group and B of FIG. 4 is for the experimental group. Compared with the control group injected with the adenovirus not carrying hM4Di gene, the sympathetic nerve activity on the side injected with the adenovirus carrying hM4Di gene was inhibited, resulting in a decrease in the number of the H-subtype bone vascular endothelial cells.

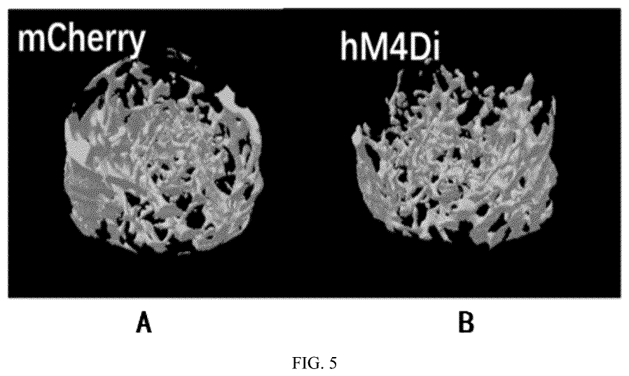

FIG. 5 shows microCT images of the trabecular bone at the epiphyseal end of the femur of the mouse, wherein A of FIG. 5 is for the control group and B of FIG. 5 is for the experimental group. Compared with the control group injected with the adenovirus not carrying hM4Di gene, the experimental group injected with the adenovirus carrying hM4Di gene showed a decrease in the bone mass of the trabecular bone after inhibition of the sympathetic nerves.

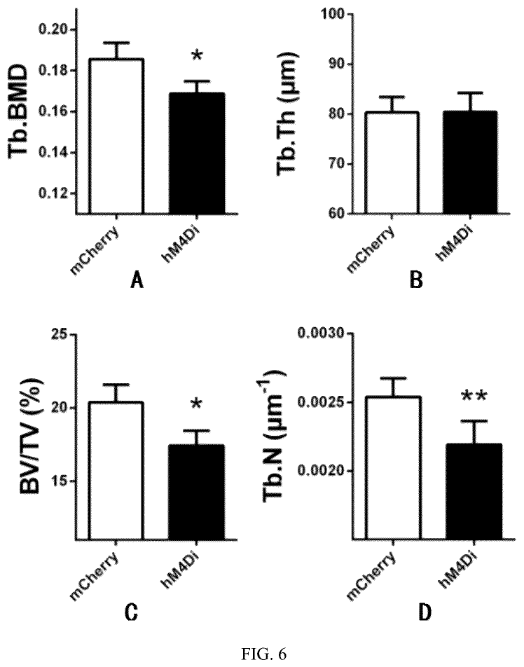

FIG. 6 shows statistical charts for the changes in the bone mass and morphology of the trabecular bone, wherein A of FIG. 6 is for the trabecular bone mineral density, B of FIG. 6 is for the trabecular bone thickness, FIG. C of 6 is for the trabecular bone volume fraction, and D of FIG. 6 is for the trabecular bone number. Compared with the control group, the experimental group showed a significant decrease in both trabecular bone mineral density and trabecular bone volume fraction, with a trend of decrease in the trabecular bone thickness and trabecular bone number.

As can be seen from the experimental results shown in FIGS. 4-6, by expressing hM4Di in the intraosseous neuron cells and injecting the designer drug CNO, the sympathetic nerve activity could be inhibited, such that the H-subtype bone vascular endothelial cells near the long bone growth plate were reduced, resulting in a decrease in the bone mass of the trabecular bone.

### Example 3

Provided in this example was a pharmaceutical composition for changing the bone mass or bone mineral density, comprising a virus carrying a chemical genetic gene and/or a designer drug activating a chemical genetic gene. The chemical genetic gene was a gene encoding an artificial designer protein receptor exclusively activated by a designer drug, and the artificial designer protein receptor could activate or inhibit the neuronal activity under the action of the designer drug. In this example, the virus was an adeno-associated virus.

In a preferred embodiment, when the artificial designer protein receptor was hM4Di, the designer drug clozapine N-oxide activated hM4Di to inhibit the neuronal activity and reduce the number of the H-subtype endothelial cells, thereby reducing the bone mineral density or bone mass.

In another preferred embodiment, when the artificial designer protein receptor was hM3Dq, the designer drug clozapine N-oxide activated hM3Dq to activate the neuronal activity and increase the number of the H-subtype endothelial cells, thereby increasing the bone mineral density or bone mass.

The preferred target subject for the pharmaceutical compositions of the above preferred embodiments in this example was a mouse aged 3-14 weeks.

### Example 4

Provided in this example was a pharmaceutical composition for treating or preventing osteoporosis, comprising a virus carrying a chemical genetic gene and/or a designer drug activating a chemical genetic gene. The chemical genetic gene was a gene encoding an artificial designer protein receptor exclusively activated by a designer drug, and the artificial designer protein receptor could activate the neuronal activity under the action of the designer drug. In this example, the virus was an adeno-associated virus.

In a preferred embodiment, the artificial designer protein receptor was hM3Dq, and the designer drug was clozapine N-oxide.

The preferred target subject for the pharmaceutical composition of the above preferred embodiment in this example was a mouse aged 3-14 weeks.

### Example 5

Provided in this example was a kit, comprising the pharmaceutical composition for changing the bone mass or bone mineral density described herein, or the pharmaceutical composition for treating or preventing osteoporosis described herein. In a preferred embodiment, the kit contained the pharmaceutical composition in Example 3 or 4.

The kit in this example could be used for constructing an animal model, for example, a mouse model of osteoporosis, and the constructed animal model could be used for experimental research on a pathogenic mechanism, a prevention method or a treatment method for osteoporosis.

In another preferred embodiment, the method for controlling the heterogeneity of the vascular endothelial cells by utilizing the sympathetic nerves, the method for changing the bone mineral density or bone mass, or the method for treating or preventing osteoporosis could also be utilized to construct an animal model, such as a mouse model of osteoporosis.

It is obvious that the above examples are merely illustrative for a clear explanation and are not intended to limit the embodiments. Various changes and modifications can be made by those of ordinary skills in the art on the basis of the above description. It is unnecessary and impossible to exhaustively list all the embodiments herein. Obvious changes or modifications derived therefrom still fall within the protection scope of the present invention.

What is claimed is:

1. A method for controlling heterogeneity of vascular endothelial cells by utilizing sympathetic nerves, comprising expressing an artificial designer protein receptor exclusively activated by a designer drug in neuron cells, and then activating the artificial designer protein receptor by the designer drug, thereby activating or inhibiting neuronal activity to achieve control of the heterogeneity of the vascular endothelial cells,
characterized in that the artificial designer protein receptor is a protein receptor that can activate or inhibit the neuronal activity under the activation effect of the designer drug.

2. The method according to claim 1, characterized in that the artificial designer protein receptor is hM4Di or hM3Dq;
the designer drug is clozapine N-oxide or deschloroclozapine;
the neuron cells are neuron cells in a young mammal.

3. The method according to claim 2, characterized in that the mammal is a mouse aged 3-14 weeks.

4. The method according to claim 1, characterized in that the vascular endothelial cells are bone vascular endothelial cells.

5. The method according to claim 4, characterized in that the artificial designer protein receptor is hM4Di, the designer drug activates hM4Di to inhibit the neuronal activity and reduce the number of H-subtype endothelial cells;
the artificial designer protein receptor is hM3Dq, the designer drug activates hM3Dq to activate the neuronal activity and increase the number of H-subtype endothelial cells.

6. A method for changing bone mineral density or bone mass, comprising expressing an artificial designer protein receptor exclusively activated by a designer drug in intraosseous neuron cells, and then activating the artificial designer protein receptor by the designer drug to activate or inhibit neuronal activity and modulate the number of H-subtype endothelial cells, thereby changing the bone mineral density or bone mass,
characterized in that the artificial designer protein receptor is a protein receptor that can activate or inhibit the neuronal activity under the activation effect of the designer drug.

7. The method according to claim 6, characterized in that the artificial designer protein receptor is hM4Di or hM3Dq;
the designer drug is clozapine N-oxide or deschloroclozapine;
the intraosseous neuron cells are intraosseous neuron cells in a young mammal.

8. The method according to claim 7, characterized in that the mammal is a mouse aged 3-14 weeks.

9. The method according to claim 6, characterized in that the artificial designer protein receptor is hM4Di, the designer drug activates hM4Di to inhibit the neuronal activity and reduce the number of the H-subtype endothelial cells, thereby reducing the bone mineral density or bone mass;
the artificial designer protein receptor is hM3Dq, the designer drug activates hM3Dq to activate the neuronal activity and increase the number of the H-subtype endothelial cells, thereby increasing the bone mineral density or bone mass.

10. A pharmaceutical composition for changing bone mass or bone mineral density, comprising an artificial designer protein receptor exclusively activated by a designer drug and/or a designer drug, or comprising a virus carrying a chemical genetic gene and/or a designer drug activating a chemical genetic gene, characterized in that the chemical genetic gene is a gene encoding an artificial designer protein receptor exclusively activated by a designer drug;
the artificial designer protein receptor is a protein receptor that can activate or inhibit the neuronal activity under the activation effect of the designer drug.

11. The pharmaceutical composition according to claim 10, characterized in that the virus is an adeno-associated virus or a lentivirus;
the artificial designer protein receptor is hM4Di or hM3Dq;
the designer drug is clozapine N-oxide or deschloroclozapine;
a target subject for the pharmaceutical composition is a young mammal.

12. The pharmaceutical composition according to claim 11, characterized in that the mammal is a mouse aged 3-14 weeks.

13. The pharmaceutical composition according to claim 10, characterized in that the artificial designer protein receptor is hM4Di, the designer drug activates hM4Di to inhibit the neuronal activity and reduce the number of H-subtype endothelial cells, thereby reducing the bone mineral density or bone mass;
the artificial designer protein receptor is hM3Dq, the designer drug activates hM3Dq to activate the neuronal activity and increase the number of H-subtype endothelial cells, thereby increasing the bone mineral density or bone mass.
